(19) United States
(12) Patent Application Publication
(10) Pub. No.: US 2009/0068643 A1
(43) Pub. Date: Mar. 12, 2009

(54) Dual Function Primers for Amplifying DNA and Methods of Use

(75) Inventors: Mark A. Behlke, Coralville, IA (US); Joseph A. Walder, Chicago, IL (US)

Correspondence Address: JOHN PETRAVICH;INTEGRATED DNA TECHNOLOGIES, INC., 8180 MCCORMICK BLVD., SKOKIE, IL, 60076-2920, US

(73) Assignee: INTEGRATED DNA TECHNOLOGIES, INC., Skokie, IL (US)

(21) Appl. No.: 11/563,072

(22) Filed: Nov. 24, 2006

Related U.S. Application Data
(60) Provisional application No. 60/739,664, filed on Nov. 23, 2005.

(51) Int. Cl. C12Q 1/68; C07H 21/00

(52) U.S. Cl. 435/6; 536/24.33; 536/23.1

(57) ABSTRACT

The present invention provides novel nucleotide compositions that enable the detection of DNA synthesis products and methods for use thereof. In one embodiment, the method can be used in PCR and allows the progress of the reaction to be monitored as it occurs. In one embodiment, the invention employs at least one fluorescence-quenched oligonucleotide that can prime DNA extension reactions. In a second embodiment, the invention employs at least one fluorescence-quenched oligonucleotide that can function as a template for DNA extension reactions. In both embodiments, the oligonucleotide also functions as a probe for detecting the progress of successive extension reaction cycles. Signal detection is dependent upon DNA synthesis and can occur with or without probe cleavage.

### FIELD OF THE INVENTION

This invention relates generally to the field of nucleic amplification and probing, and more particularly, to methods and compositions for performing PCR and probe hybridization using a single reagent mixture.

### BACKGROUND OF THE INVENTION

The polymerase chain reaction (PCR) has become almost essential for the efficient execution of techniques ranging from cloning, analysis of gene expression, DNA sequencing, and genetic mapping, to drug discovery, criminal forensics, and the like. (Mullis, et al., Cold Spring Harbor Symp. Quant. Biol. 51:263-273 (1986); Saiki, et al., Science 230:1350-1354 (1985); Innis et al. in PCR Protocols A guide to Methods and Applications, Academic Press, San Diego (1990); and U.S. Pat. Nos. 4,683,195, 4,683,202). Originally PCR amplification and amplification product detection were performed separately. More recently, this process has been improved by combining these steps into a single reaction mixture that contains both PCR reagents and probing reagents. This improvement allows for the incorporation of all reagents at once so that products can be generated and detected without ever opening the reaction tube. This improvement has reduced the opportunity for cross-contamination between samples and has reduced the number of manipulations and time required to obtain the result of an experiment.

A number of methods now exist for detecting PCR amplification products as they are generated (“real-time” PCR). In general, these methods employ a fluorescence-quenched probe in which a fluorescent reporter dye is linked to an oligonucleotide that also contains a quencher group such that the fluorescence of the oligonucleotide is quenched when it is added to an amplification reaction mixture. The oligonucleotide is designed to selectively hybridize to amplified target DNA, i.e. “target specific” oligonucleotide. A fluorescent signal is generated as the quenching of the fluorescent reporter is reduced by a variety of mechanisms all of which require interaction of the probe with amplified target sequences.

In one “real time” PCR method an oligonucleotide probe that is non-extendable at the 3′ end, is labeled with a fluorophore at its 5′ end and a quencher so that the quencher quenches the fluorescence of the fluorophore. Hybridization of the probe to its target sequence during amplification generates a substrate suitable for cleavage by the exonuclease activity of the PCR polymerase. During amplification, the 5→3′ exonuclease activity of the polymerase enzyme degrades the probe into smaller fragments. When a site between the quencher and fluorophore is cleaved, the fluorophore and quencher become more spatially separated and quenching is lost. This gives rise to a fluorescent signal. This assay has come to be known as the Taqman® assay. While this method provides a significant improvement over prior methods that required a separate detection step, the assay has some drawbacks. Namely, the assay requires the synthesis of at least three target specific oligonucleotides despite the fact that only two oligonucleotides are needed for amplification. The amplification reaction assay also requires a polymerase that has a 5→3′ exonuclease activity that can efficiently digest fluorophore/quencher labeled oligonucleotide probes.

Linear, dual-labeled, fluorescent-quenched oligonucleotide probes can also be modified at the 5′ end such that exonuclease degradation does not occur during PCR. Such probes are quenched in the single-stranded random coil conformation but fluoresce when in the more extended double stranded state. These probes can be included in PCR reactions and generate a fluorescent signal if and when their target sequences become amplified. Although this method eliminates the requirement for a 5→3′ exonuclease activity, the method does require three target specific oligonucleotides to carry out the amplification with “real time” detection.

Alternatively, a probe has been developed that is capable of forming a hairpin that has, within the loop of the hairpin, a sequence that is hybridizable to a target nucleic acid. The probe also includes covalently attached fluorophore and quencher molecules positioned on the oligonucleotide so that when the oligonucleotide adopts the hairpin conformation, the fluorescence of the fluorophore is quenched by the quencher. When the probe forms a duplex with its target sequence, the hairpin is disrupted and the fluorophore and quencher become spatially separated and a fluorescent signal is observed. Because the probe need not be degraded to generate a signal, this method overcomes the requirement of the previously described Taqman® assay that the polymerase have a 5→3′ exonuclease activity. Nevertheless, as with the previously described assays, this method requires three target sequence specific oligonucleotides. In addition, it limits the possible probe sequences to those capable of forming hairpin structures. Not only does the hairpin sequence interfere with the kinetics and thermodynamics of probe-target binding but such structures can be difficult to chemically synthesize.

One “real time” amplification detection method eliminates the requirement for three target specific primers. In this method, the 5′ end of an amplification primer contains an oligonucleotide extension. The extension contains a fluorophore and quencher and can adopt a hairpin conformation such that fluorescence is quenched in the isolated primer. Once the primer is incorporated into a double stranded amplicon, and the hairpin on the 5′-end of the primer-probe is disrupted, the fluorophore becomes spatially separated from the quencher and a fluorescent signal develops. Variants of this system allow the hairpin structures to be linked to PCR primers via covalent spacer/linker moieties.

Each of the foregoing “real time” PCR methods is limited in that they either require three oligonucleotides and/or the probes contain hairpin loops that contribute to difficulties in both probe design and synthesis and compete with duplex formation with amplified DNA strands. New methods are needed that require only two target specific oligonucleotides for amplification and “real-time” detection of amplified products. To facilitate probe design and synthesis and to eliminate the competition between hairpin and duplex formation, such methods should also avoid the use of hairpin loop structures.

The invention provides such compositions and methods. These and other advantages of the invention, as well as additional inventive features, will be apparent from the description of the invention provided herein.

### BRIEF SUMMARY OF THE INVENTION

The present invention provides a novel nucleotide composition that enables the detection of DNA synthesis products and methods for use thereof. In one embodiment, the method can be used in PCR and allows the progress of the reaction to be monitored as it occurs. The invention employs at least one fluorescence-quenched oligonucleotide that can prime DNA extension reactions. In addition to priming extension reactions, the oligonucleotide also functions as a probe for detecting the progress of successive extension reaction cycles.

### BRIEF DESCRIPTION OF THE DRAWINGS

FIG. 1 is a diagram illustrating the stages of real-time detection during amplification using a first primer having a 3′ target binding domain and a 5′-template-probe binding domain. The 3′-target binding domain is specific to the target, containing sufficient complementarity to bind to the target under standard conditions employed in PCR, and can function as a primer in PCR. The 5′-template probe binding domain is not complementary to the target and instead is complementary to a synthetic template-probe nucleic acid.

FIG. 2 is a graph of real-time spectrofluorometric plots of the PCR assays using a fluorescence/quenched probe/primer with in standard Taqman® reaction buffers and cycle parameters.

FIG. 3a shows the fluorescence signal, and FIG. 3b shows the signal to noise ratio of the duplex assays detailed in Example 2. In FIG. 3a the bars in the three bar set, from left to right, represent the fluorescence observed from a single stranded primer/probe, the corresponding duplex primer/probe, and the corresponding Micrococcal Nuclease digested primer/probe. FIG. 3b shows a series of two bars for oligonucleotides at each quencher-fluorophore spacing.

FIG. 4 is a graph of “real-time” spectrofluorometric plots of the PCR assay to test whether the observed signal to noise data correlates with functional performance of a primer/probe.

FIGS. 5a and 5b are graphs depicting the function ability of TAMRA-containing probes.

FIG. 6 is a photograph of a gel having three lanes that indicate where the Tli I enzyme was added either pre-PCR, post-PCR (additional 30′ incubation at 75° C.), or not added. Products were separated using PAGE (10% gel, denaturing conditions), stained using Gelstar, and visualized with UV. From left to right the first two lanes show that cleavage occurred whether Tli I was added to reactions either before PCR (lane 1) or after PCR (lane 2). The third lane shows full length, uncleaved product when no Tli I was added.

FIG. 7 is a diagram of the spatial relationship between the probes, target and template of an FQT assay described in Example 8.

FIG. 8 is an amplification plot demonstrating the efficacy of an FQT assay with or without cleavage using PspG1 and with or without forward primer. The assay is dependent on the presence of the forward primer. The results demonstrate that the assay obtains a slightly better signal with cleavage by the PspG1 enzyme.

FIG. 9 is an amplification plot demonstrating the efficacy of an FQT assay with or without cleavage using PspG1 and with or without chimeric reverse primer. The assay is dependent on the presence of chimeric reverse primer.

FIG. 10 is an amplification plot illustrating the efficacy of the FQT assay format as compared to a 5′-nuclease assay. Although the 5′-nuclease assay has a slightly stronger signal, both assays show a similar sensitivity.

FIG. 11 is an amplification plot illustrating the efficacy of the FQT assay format as compared to a FQ assay. The FQ assay emits a slightly stronger signal but both assays demonstrate similar sensitivity.

FIG. 12 is an amplification plot illustrating comparing FQT assays containing LNA and 5-methyl-dC modifications. The LNA-modified probes have a stronger signal, but both assays demonstrate similar sensitivity.

FIG. 13 is an amplification plot comparing 5-methyl-dC probes with and without enzymatic cleavage. The cleavage format emits a slightly stronger signal.

### DETAILED DESCRIPTION OF THE INVENTION

The oligonucleotide contains two functional domains, a primer domain and a fluorescence-quenched reporter domain. The primer domain has complementarity to a desired target sequence and functions to prime PCR or other DNA extension reactions. This domain can be comprised of modified or unmodified DNA and is located at the 3′-end of the oligonucleotide. The reporter domain also contains DNA bases but is modified to contain both a fluorophore (reporter) group and a quencher group and is located at the 5′-end of the oligonucleotide. This domain may or may not be complementary to the template. The reporter domain does not comprise any nucleic acid sequence or structure that would lead to formation of a hairpin or other stable secondary structure that forces reporter and quencher into contact. While the primer domain functions to prime DNA synthesis, both primer and reporter domain can function as a template for DNA synthesis such that, during the process of repeated cycles of DNA synthesis, the oligonucleotide is converted from single-stranded to double-stranded form. In all embodiments, the fluorescence of the oligonucleotide is quenched in the single-stranded form (prior to priming DNA synthesis). This is achieved by interaction between reporter and quencher in random coil conformation.

Various embodiments are contemplated that differ in the mechanism of signal generation (i.e., release of fluorescence quenching). Preferably, each variant employs slightly different probe designs. One embodiment measures the increase in fluorescence signal that occurs with the transition from single-stranded DNA to duplex DNA during DNA synthesis or PCR. The end-to-end distance between points on a DNA molecule is shorter for random coil conformation single stranded DNA than for more rigid duplex DNA. If spacing between reporter and quencher is chosen so that the single stranded form falls within the Förster radius for the reporter/quencher pair and the duplex form exceeds the Förster radius (the Förster radius is unique for each reporter/quencher combination), then the single stranded form will be quenched while the duplex form will not be quenched. Therefore, the only event needed to release quenching and produce fluorescence signal is formation of duplex DNA (hereafter referred to as “FQ uncleaved”). The signal is not achieved simply by hybridization to target, but rather the method of the invention achieves duplex formation by DNA synthesis, where the probe itself serves as one primer. In this way signal generation is directly linked to DNA synthesis so that in PCR detectable fluorescence will accumulate with each reaction cycle and can be monitored as strands accumulate. Alternatively, fluorescence signal can be measured at the completion of PCR.

Another embodiment of the method measures the increase in fluorescence signal that occurs when the reporter and quencher are separated by cleavage of intervening bases by action of a nuclease. This method again requires that the probe/primer be in duplex form, preferably as a result of a DNA synthesis or PCR reaction wherein the probe/primer itself functions as a primer. Any nuclease that cleaves double-stranded nucleic acid to result in separation of reporter and quencher falls within the scope of the invention (hereafter referred to as “FQ cleaved”. Two specific examples are described.

One method to separate reporter and quencher by nuclease action is to cleave the DNA between groups using a restriction endonuclease. In general, restriction endonucleases do not cleave single-stranded DNA but require a double-stranded DNA substrate. In this way the restriction endonuclease will not cleave the original probe/primer oligonucleotide and the enzyme can be present during DNA synthesis or PCR. When the probe/primer become double-stranded following DNA synthesis, it becomes a substrate for the restriction endonuclease and will be cleaved. Cleavage separates reporter from quencher and a fluorescence signal can be detected, such that signal generation is directly linked to DNA synthesis and can be followed in real time during DNA synthesis. If the restriction enzyme employed is thermostable, then DNA synthesis and probe cleavage can progress simultaneously in the same reaction during PCR. For example, one suitable thermostable DNA restriction endonuclease is Tli I (New England Biolabs, Beverly, Mass.). The recognition site for Tli I is “CTCGAG”; if this sequence is positioned between the reporter group and the quencher group, then Tli I can cleave the probe (in duplex form). A variety of thermostable restriction endonucleases have been identified, many of which may be suitable for use. Restriction endonucleases that are not thermostable can be used after PCR is complete as an end-point assay.

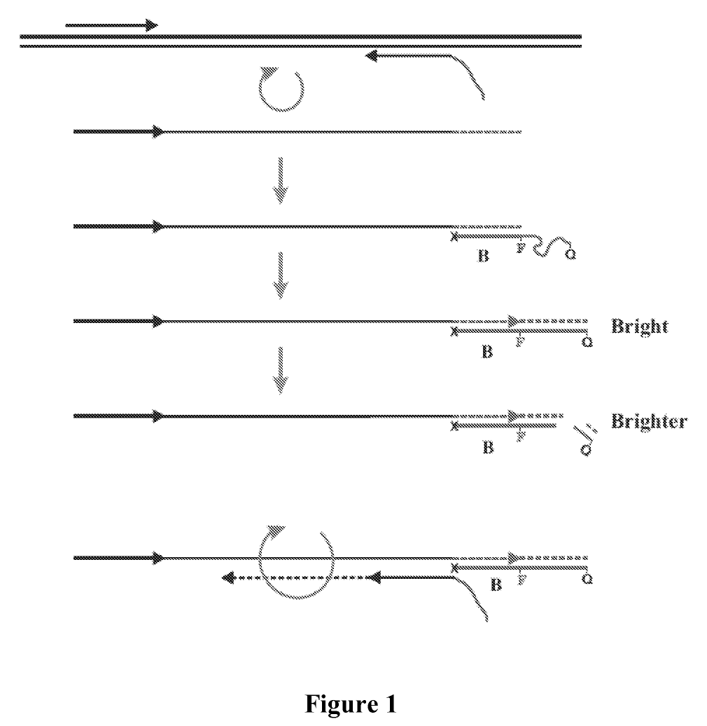

In another embodiment, summarized by the diagram in FIG. 1, the assay (hereafter referred to as “FQT” to differentiate between the prior “FQ” embodiments) uses a first primer having a 3′-target binding domain and a 5′-template-probe binding domain. The 3′-target binding domain is specific to the target, containing sufficient complementarity to bind to the target under standard conditions employed in PCR, and can function as a primer in PCR. The 5′-template probe binding domain is not complementary to the target and instead is complementary to a synthetic template-probe nucleic acid.

In a first primer extension reaction, the initial extension product formed comprises the probe binding domain at its 5′ end; the source of this domain is from the PCR primer. In the next primer extension reaction (cycle 2 of PCR), a complement of the first extension product comprising the complement of the probe binding domain at its 3′ end is synthesized. A complementary copy of the template-probe specific sequence is now joined to target sequence on the other strand via DNA synthesis, using the original primer as template. In this fashion, target-template sequence becomes linked to template-probe sequence. It will be appreciated that now the template-probe domain is on the 3′-end of the newly synthesized DNA strand and is now competent to itself serve as a primer in subsequent PCR reactions.

A template-probe comprising at its 3′ end a sequence complementary to the probe binding domain is hybridized to the 3′ end of the second extension product. The probe comprises a 5′ region that does not hybridize to the second extension product in which there is both a fluorophore moiety and a quencher moiety. When the portion of the probe comprising the fluorophore moiety and quencher moiety is single stranded, fluorescence is quenched. The template-probe is blocked at the 3′-end so this nucleic acid cannot serve as a primer. One suitable blocking group for this purpose is dideoxycytidine (ddC).

In a third primer extension reaction, the second strand is extended such that a complement to the 5′ region of the probe is synthesized. The probe thus becomes at least partially double stranded. Formation of duplex DNA by DNA synthesis extends the distance between fluorophore and quencher resulting in an increase in fluorescence (hereafter referred to as “FQT uncleaved”. Optionally, the probe is designed to include a nuclease susceptible sequence between reporter and quencher. Many different cleavable elements could be placed at this location. As one example, a restriction endonuclease restriction site, which when cleaved by said nuclease, results in physical separation of reporter and quencher, thereby leading to a further increase in fluorescence intensity (hereafter referred to as “FQT cleaved”). One suitable restriction endonuclease recognition site is CC(A/T)GG which is cleaved by the thermophilic restriction enzyme PspG1. This process can be repeated with subsequent rounds of amplification.

FIG. 1 demonstrates that if the binding domain of the template has a high enough Tm, all reactions shown in FIG. 1 can run concurrently in real time. Residues such as 5-methyl-dC (5Me-dC), 5-propynyl-dC (pdC), or locked nucleic acids (LNA's) may be incorporated within the binding domain (“B”) of the template probe to increase Tm. The “x” represents a blocking group on the 3′-end of the template-probe which serves to prevent the template from itself priming DNA synthesis.

In this embodiment of the invention, the fluorescence-quenched template oligonucleotide does not have any sequence domains complementary to target. The FQT template component of the detection reaction can therefore serve as a universal detection reagent which can be employed in detection assays for any number of different nucleic acid target sequences. The target-specific components of this reaction reside in oligonucleotide primers which can be synthesized without the inclusion of costly modifications, such as fluorophore or quencher groups. The modified FQT probe can be manufactured more economically in large scale and used as the detection reagent for multiple reactions whose specificity is determined by inexpensive, unmodified oligonucleotide primers.

Another method to separate reporter and quencher by nuclease action is to position RNA bases between the reporter and quencher groups and cleave using RNase H. RNase H is an endoribonuclease that specifically cleaves the RNA portion of an RNA/DNA heteroduplex and does not cleave single-stranded RNA. The cleaved nucleic acid does not have to be entirely composed of RNA. Preferably, it can be a chimera that contains both RNA and DNA residues, however cleavage occurs within the RNA segment. In one embodiment, the RNA content will include at least 4 consecutive RNA residues, which constitutes a fully active substrate for RNase H. Thus the primer/probe oligonucleotide for this method will be a DNA/RNA chimera wherein around 4 RNA bases are positioned as a consecutive grouping between the reporter and quencher. While RNA cannot generally be used as a template for DNA synthesis with most polymerases (other than reverse transcriptase), short stretches of RNA can be inserted in chimeras and will function with many DNA polymerase enzymes. Thus the chimeric RNA/RNA probe/primer can function both as primer, template, and probe. Further, thermostable RNase H is available, enabling a homogenous assay format where DNA synthesis or PCR occurs simultaneously with probe cleavage.

In another embodiment, a variation of RNase H cleavage is employed wherein cleavage occurs at a single ribonucleotide base in a DNA sequence. As outlined previously, one substrate for RNase H is an RNA nucleic acid in an RNA/DNA heteroduplex with cleavage occurring at the 3′-end following a central RNA residue, leaving a free 3′-OH. Certain members of the RNase H family of enzymes have the capacity to cleave other substrates. For example, one class of enzyme can cleave a nucleic acid molecule that has a single RNA residue in a DNA sequence when annealed in double-strand conformation with DNA. In this case, cleavage occurs 5′ to the RNA residue and again leaves a free 3′-OH. The human RNase H1 enzyme was demonstrated to cleave such a substrate (Eder et al., J. Biol. Chem. 266 (1991), 6472-6479). Similar RNase H enzymes have been discovered in mice (see Cerritelli et al., Genomics 53 (1998), 300-307 for mouse RNase H1) and in prokaryotes (see Haruki et al, FEBS Letters 531 (2002) 204-208 for RNases HII from Bacillus subtilis and Thermococcus kodakaraensis). A thermophilic RNase H capable of cleaving a heteroduplex containing a single ribonucleotide could be used in the proposed assay and permit cleavage and detection to take place in real time concurrent with amplification. Cleaving with an RNase H-type enzyme could be utilized in FQ or FQT cleaving embodiments.

The following set of restriction enzymes are commercially available and would appear to satisfy the requirement that the enzyme be stable at elevated temperatures. The enzymes Tli I and PspG I are derived from “extreme” thermophiles and will survive conditions used in PCR. The remaining enzymes have been identified by the manufacturers as stable for 20′ at 80° C.

| | | Suggested |
|---|---|---|
| | Recognition | reaction |
| Enzyme | Sequence | temperature |
| Bcl I | \| | 50° C. |
| | . . T GATCA . . | |
| BstB I | \| | 65° C. |
| | . . TT CGAA . . | |
| BstE II | \| | 60° C. |
| | . . G GTNACC . . | |
| BstN I | \| | 60° C. |
| | . . CC (A/T)GG . . | |
| BstU I | \| | 60° C. |
| | . . CG CG . . | |
| Mwo I | \| | 60° C. |
| | . . GCNNNNN NNGC . . | |
| PspG I | \| | 75° C. |
| | . . CC(A/T)GG . . | |
| Sfi I | \| | 50° C. |
| | . . GGCCNNNN NGGCC . . | |
| Sml I | \| | 55° C. |
| | . . C TYRAG . . | |
| Tfi I | \| | 65° C. |
| | . . G A(A/T)TC . . | |
| Tli I | \| | 75° C. |
| | . . C TCGAG . . | |
| Tse I | \| | 65° C. |
| | . . G C(A/T)GC . . | |
| Tsp45 I | \| | 65° C. |
| | . . GT(G/C)AC . . | |
| Tdp509 I | \| | 65° C. |
| | . . AATT . . | |
| TspR I | \| | 65° C. |
| | . . NNCA(C/A)TGNN . . | |
| Tth111 I | \| | 65° C. |
| | . . GACN NNGTC . . | |

Note: Tli I is a thermostable isoschizomer of Xho I.

The various embodiments of the proposed invention can work in a number of amplification methods well-known in the art. The proposed invention can work in polynomial amplification (see Behlke et al., U.S. Pat. No. 7,112,406). Polynomial amplification (“polyamp”) reactions, as described in Behlke et al., employ oligonucleotide primers in one direction (“forward” primer) that are modified at internal position(s) in a way that blocks their function when they serve as a template while they retain their primer activity (i.e., are “replication defective” primers). The second (“reverse”) primer is “replication competent” and generally is unmodified. Multiple replication defective primers can be used together in a nested fashion to increase the amplification power of the reaction. Generally a single replication competent reverse primer is used.

A variety of products are made during polynomial amplification, the precise nature of which depends upon the number of nested replication defective forward primers employed. While each product varies in length, they all share one end in common which is defined by the single reverse primer. Opposing ends are defined by the blocking domain for each modified forward primer employed.

Accurate detection methods of polynomial amplification are limited. A 5′-nuclease assay detects a variety of products and is not specific for the terminal polyamp reaction product. The FQT assay provides a more accurate assay format. The method involves annealing an oligonucleotide (“polyamp FQT probe”) to the 3′-end of the terminal amplification product. The annealed oligonucleotide serves as a template for a DNA synthesis reaction using the amplification product as a primer. A primer extension reaction is performed in the presence of unlabeled dNTPs and can take place concurrently with amplification in the same tube. An amplification product having a 3′-end which is complementary to the binding domain of the FQT probe is required for this reaction to proceed. This product specifically results from polyamp where the reaction product terminates in the blocking domain of the innermost replication defective primer. This new detection scheme confers the following two added levels of specificity to the detection event: 1) specific hybridization must occur between the detection template oligonucleotide and the polyamp product, and 2) an amplified product must be present that has a free 3′-end available to prime DNA synthesis when coupled to the above hybridization event.

The following examples further illustrate the invention but, of course, should not be construed as in any way limiting its scope.

### EXAMPLE 1

This example demonstrates that fluorescence-quenched primer/probes can be used to amplify target DNA and give a “real-time” fluorescent signal corresponding to the quantity of amplified target DNA.

The following oligonucleotides were prepared for this example:

| SEQ. | | |
|---|---|---|
| ID | | |
| NO. | Sequence | Notes |
| 1 | CCAGCCGTAGTCGGTAGTAATCTATCAA | target |
| | GTTCTCATCGAAGCGGATAGGCGAGCG | |
| 2 | CCAGCCGTAGTCGGTAGT | PCR primer |
| | | “for” |
| 3 | CGCTCGCCTATCCGCTTC | PCR primer |
| | | “rev” |
| 4 | AQ-CCGTTCTCGAGTTtCGCTCGCCTAT | FQ probe- |
| | CCGCTTC | primer (rev) |

SEQ ID NO: 1 served as a target for amplification. SEQ ID NOS: 2, 3 and 4 were used to amplify the target. SEQ ID NO: 4 had the same priming sequence as SEQ ID NO: 3 and also contained an additional nucleotide sequence on its 5′-end. In SEQ ID NO: 4 the additional nucleotide sequence contained a fluorophore and quencher but the structure did not have a sequence that would lead to hairpin loop formation. The fluorescein modified dT base is denoted t. The fluorophore was fluorescein and was added to the oligonucleotide as fluorescein-dT using known phosphoramidite chemistry in an automated synthesizer. The quencher was a proprietary anthraquinone quencher described in U.S. patent application Ser. No. 10/666,998 which was added to the 5′-terminal hydroxyl group using standard phosphoramidite chemistry in an automated synthesizer. The linkage of the anthraquinone quencher to the oligonucleotide is shown below in FIG. 1.

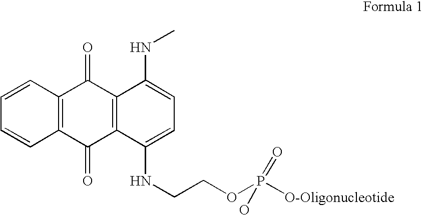

For all sequences A, C, G, and T represent deoxynucleotides (DNA) and the oligonucleotide sequences are written with the 5′ end to the left and the 3′ end to the right, unless otherwise noted. Oligonucleotide substrates were synthesized using standard phosphoramidite chemistry on an Applied Biosystems Model 394 DNA/RNA synthesizer.

Following synthesis, oligonucleotides were cleaved from the solid support and deprotected using standard methods. Oligonucleotides were then purified by reverse-phase HPLC with a Hamilton PRP-1 column (1.0 cm×25 cm) using a linear gradient of from 5 to 50% acetonitrile in 0.1 M triethyl-ammonium acetate (TEAAc) at pH 7.2 over 40 min. Monitoring was at 260 nm and 494 nm and fractions corresponding to the fluorescent-labeled oligonucleotide species were collected, pooled, and lyophilized. Oligonucleotides were dissolved in 200 μl of sterile water and precipitated by adding 1 ml of 2% LiClO₄, followed by centrifugation at 10,000 g for 10 min. The supernatant was decanted and the pellet washed with 10% aqueous acetone.

Oligonucleotides were further purified by ion exchange HPLC using a 40 min linear gradient of 0% to 50% 1 M LiCl in 0.1 M TRIS buffer over 40 min. Monitoring was at 260 nm and 494 nm and fractions corresponding to the dual-labeled oligonucleotide species were collected, pooled, precipitated with 2% LiClO₄, and lyophilized. Oligonucleotide identities were verified by mass spectroscopy using a Voyager-DE BioSpectrometry workstation. Once verified the oligonucleotides were used in PCR reactions.

PCR reaction mixtures had the following compositions in a 25 μl reaction volume:

### 20 mM TrisHCl pH 8.3,

### 50 mM KCl,

### 5 mM MgCl₂,

200 nM each dNTP
200 nM PCR primer “for”
200 nM PCR primer “rev” or “PCR probe-primer”
10², 10⁴, 10⁶ and 10⁸ copies Target DNA
2.5 units AmpliTaq Gold DNA Polymerase

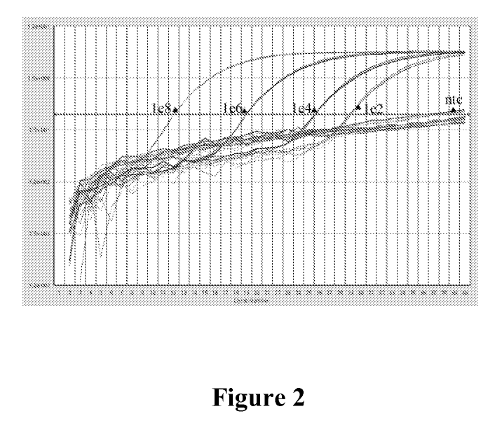

Reaction mixtures were initially treated at 95° C. for 10 min. Then a two-step PCR cycle was used, wherein the target was denatured at 95° C. for 15 sec., followed by annealing and extension at 60° C. for 60 sec. Real-time spectrofluorometric plots of the PCR assays are shown in FIG. 2.

As shown in FIG. 2, the dual-labeled primer, SEQ ID NO: 4, efficiently primed amplification of the target sequence and provided increasing fluorescent signals as the amplification progressed. The sample with the highest concentration of target, 10⁸ copies, had the most rapid exponential increase in fluorescence (i.e., lowest Ct value) which occurred at 12 cycles. The reaction with 10⁶ copies of target had a Ct value of 19 cycles, the reaction with 10⁴ copies of target had a Ct value of 25 cycles and the 10² target reaction had a Ct value of 29 cycles. All samples generated a similar maximum signal by the end of 40 cycles. Also shown, for comparison, is the fluorescence of reactions in which no amplification occurred.

This example demonstrates that the inventive dual-labeled primers can be used to amplify target nucleic acid sequences and, simultaneously, provide a direct signal for monitoring the progress of amplification. This example also shows that target numbers as low as 100 copies can be efficiently amplified with these primers. In this embodiment, no probe cleavage occurred. The signal is generated from the release of quenching as the probe is converted to double-stranded DNA. Probe cleavage and degradation are not involved.

### EXAMPLE 2

This demonstrates one method for optimizing the distance between a quencher and fluorophore so that a maximum signal to noise ratio is obtained in primer/probes. The same optimization results will apply to FQT template probes.

The fluorescence of oligonucleotide primer/probes was determined for an oligonucleotide primer/probe in three distinct physical states, namely, single-stranded, double-stranded, and after cleavage at a point between the reporter and quencher. For oligonucleotide cleavage the cleavage was carried out in two ways, first single stranded primer/probes were digested with a mixture of Micrococcal Nuclease and DNase I. Fluorescence was measured in a Tecan plate fluorometer or in a PTI cuvette fluorometer according to manufacturer's instructions.

The following oligonucleotides were studied in this example:

| | | |
|---|---|---|
| | | Fluorophore/ |
| SEQ. | | Quencher |
| ID | | Separation |
| NO. | Sequence | (bases) |

| | | |
|---|---|---|
| 5 | AQ-CCGTTtCGCTCGCCTATCCGCTTC | 6 |
| 6 | AQ-CCGTTCTtCGCTCGCCTATCCGCT | 8 |
| | TC | |
| 7 | AQ-CCGTTCTCGtCGCTCGCCTATCCG | 10 |
| | CTTC | |
| 8 | AQ-CCGTTCTCGAGtCGCTCGCCTATC | 12 |
| | CGCTTC | |
| 9 | AQ-CCGTTCTCGAGGTtCGCTCGCCTA | 14 |
| | TCCGCTTC | |
| 10 | AQ-CCGTTCTCGAGGTTTtCGCTCGCC | 16 |
| | TATCCGCTTC | |
| 11 | AQ-CCGTTCTCGAGGTTTTTtCGCTCG | 18 |
| | CCTATCCGCTTC | |
| 12 | AQ-CCGTTTTCTCGAGGTTTTTtCGCT | 20 |
| | CGCCTATCCGCTTC | |

A 400 nM solution of each oligonucleotide was prepared in 10 mM Tris pH 8, 5 mM MgCl₂. The fluorescence of each oligonucleotide was measured in this single-stranded form. Each oligonucleotide was then mixed with a two-fold molar excess of its complementary DNA, allowed to form duplexes, and fluorescence was re-measured. An aliquot of single stranded oligonucleotide was also treated with 5 units of Micrococcal Nuclease and 5 units DNase I at 37° C. for 1 h and fluorescence was measured. The Micrococcal Nuclease digest shows the maximum amount of fluorescence that can be expected from a primer/probe while the single stranded form of the oligonucleotide shows the background fluorescence of the same primer/probe.

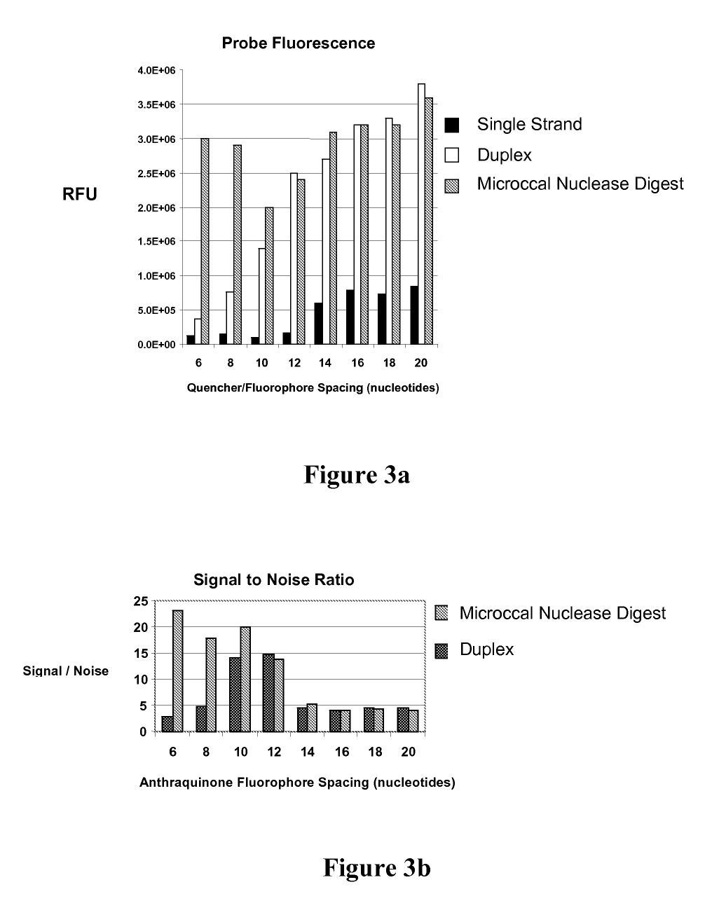

The results from these measurements are shown in FIGS. 3a and 3b in bar graph form. In FIG. 3a, the bars in the three bar set, from left to right, represent the fluorescence observed from a single stranded primer/probe, the corresponding duplex primer/probe, and the corresponding Micrococcal Nuclease digested primer/probe.

As shown in FIG. 3a, the single stranded form of the primer/probe has relatively low background fluorescence until the space between the quencher and fluorophore is about 14 nucleotides. Background fluorescence increased steadily as the separation increased beyond about 14 nucleotides. This could reflect reduced quenching efficiency resulting from the greater distance between quencher and fluorophore moieties in the single stranded random coil conformation.

The maximum fluorescent signal, observed with primer/probes digested by Micrococcal Nuclease was relatively constant for all probes. The minor differences in fluorescence observed between samples may result from variations in oligonucleotide quality. The fluorescent signal for the duplex form of the primer/probe steadily increased to the maximum as base spacing increased to about 16 base pairs.

Signal to noise ratios were calculated and are shown in bar graph form in FIG. 3b. FIG. 3b shows a series of two bars for oligonucleotides at each quencher-fluorophore spacing. The bar on the left shows a signal to noise ratio calculated by dividing the fluorescence observed with the single-stranded primer/probe into the fluorescence observed in its duplex form. The bar on the right shows a theoretical maximum signal to noise ratio which was determined for each primer/probe in the degradative Micrococcal Nuclease assay by dividing the fluorescence observed with the single-stranded primer/probe into the fluorescence observed with the digested duplex form.

For degradative assays, the signal to noise ratio is relatively high, about 15 to 20, until the distance between fluorophore and quencher rises above 12 nucleotides and then it is substantially reduced to about 5. In contrast, the duplex non-degradative assay shows a peak signal to noise ratio when the quencher and fluorophore are separated by about 12 base pairs and then abruptly declines to a minimum of about 5 when the spacing is about 14 or more nucleotides. At the shorter separation distances of about 6-8 nucleotides, peak signal intensity appears compromised because appreciable quenching exists in the duplex form. At longer separation distances of 14 or more nucleotides, peak signal intensity is achieved in the duplex form but quenching in the single stranded form is incomplete.

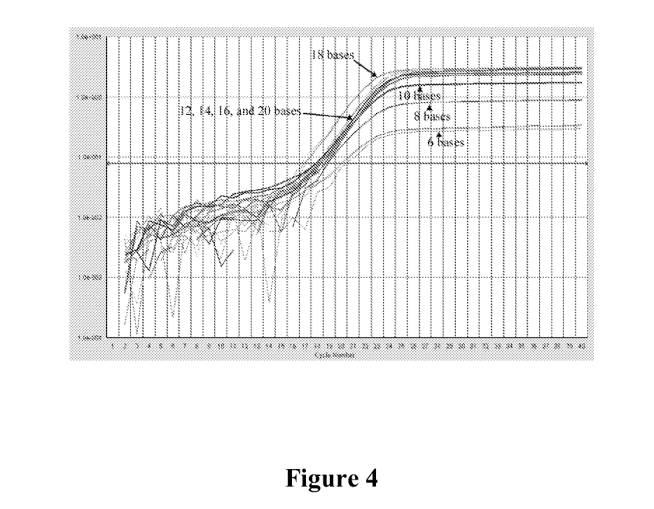

To test whether the observed signal to noise data correlates with functional performance of a primer/probe, the same probe series was used in a “real-time” PCR assay. The assay design was identical to that used in Example 1 with the primer/probe. The results from “real-time” PCR experiments with these probes is shown in FIG. 4. All probes with a quencher fluorophore separation of 12 bases or more performed equally well. The performance in the assay showed a greater correlation with peak fluorescence intensity than with the signal to noise ratio.

Thus it appears that this method can be used to optimize the spacing between the fluorophore and quencher to achieve a maximum signal to noise ratio. In this example, it appears that with fluorescein and the anthraquinone quencher the optimal spacing is about 10-12 nucleotides. Further, all probes generated a signal in the “real-time” PCR assay however, better results were obtained when the spacing between the anthraquinone quencher and fluorescein was at least 12 nucleotides. There is no need to use a restriction endonuclease cleavage if the optimal spacing between fluorophore and quencher is used. If spacing of less than 12 bases is desired, then cleavage may be a better alternative.

### EXAMPLE 3

This example shows that primer/probes can be prepared with the fluorophore TAMRA. The following oligonucleotides were prepared using the same methods as described in Example 2 with the exception that the fluorophore, TAMRA-dT, was substituted for Fluorescein-dT.

| SEQ. | | Fluorophore/ |
|---|---|---|
| ID | | Quencher |
| NO. | Sequence | Separation |
| 13 | AQ-CCGTTCTCGiCGCTCGCCTATCCGCTTC | 10 |
| 14 | AQ-CCGTTCTCGAGGTiCGCTCGCCTATCCG | 14 |
| | CTTC | |
| 15 | AQ-CCGTTCTCGAGGTTTTTiCGCTCGCCTA | 18 |
| | TCCGCTTC | |

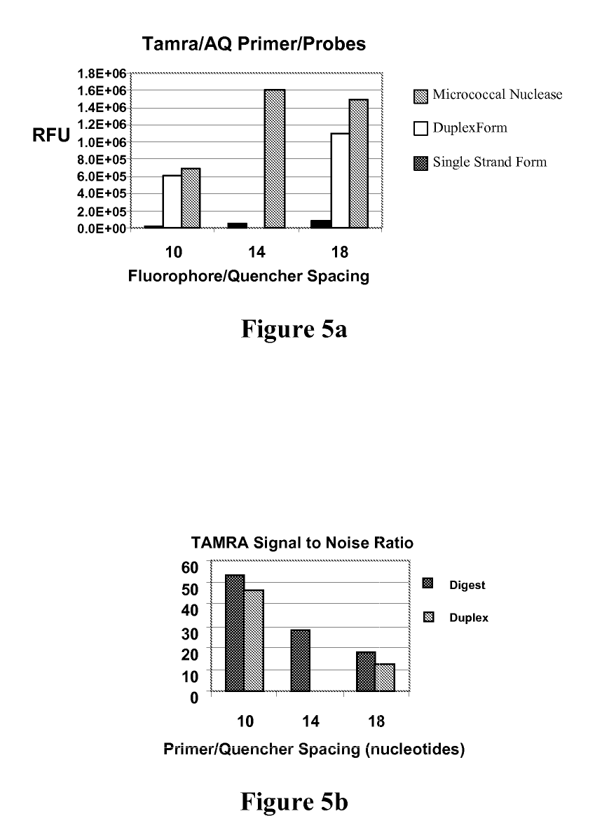

As in Example 2, the fluorescence of the oligonucleotide primer/probes was determined for an oligonucleotide primer/probe in three distinct physical states, namely, single-stranded, double-stranded, and after cleavage of the oligonucleotide between the reporter and quencher. For oligonucleotide cleavage the cleavage was carried out through digestion with a mixture of Micrococcal Nuclease and DNase I. Fluorescence was measured in a Tecan plate fluorometer or in a PTI cuvette fluorometer according to manufacturer's instructions. The results are shown in FIGS. 5a and 5b.

In general, the results obtained with TAMRA appeared remarkably similar to those obtained previously in Experiment 2 where fluorescein was used. With a ten nucleotide spacing between TAMRA and the anthraquinone quencher, there is little fluorescence of the single strand primer/probe and substantial fluorescence of the duplex and Micrococcal Nuclease digested samples. At 18 nucleotides, the background begins to rise but the duplex and Micrococcal Nuclease digested samples both demonstrate substantial fluorescence.

When used in “real-time” PCR, the primer/probes all performed equally well with their fluorescein containing counterparts from Example 2.

This example shows that the primer/probes of the invention can contain a variety of fluorophores and that design parameters of the primer/probes are not significantly affected by the choice of fluorophore. Further, this example demonstrates that TAMRA, which produces a more intense signal than Fluorescein, is an effective substitute for fluorescein in the dual-labeled probe invention.

### EXAMPLE 4

This example demonstrates that a restriction endonuclease enzyme can function in a PCR environment.

If the spacing is optimal as demonstrated in Examples 2 and 3, there is no need to utilize cleavage for separation of the fluorophore and quencher. If the spacing is less than the optimal range, then enzymatic cleavage is an alternative method and may in fact be preferred.

The dual-labeled primer/probe, SEQ ID NO: 4, in single-stranded, random-coil conformation, is not a substrate for a restriction enzyme. However, during amplification, the primer/probe is incorporated into an oligonucleotide strand and becomes double-stranded after a subsequent round of amplification. A cleavage event can occur once the primer/probe becomes incorporated into a duplex structure. By positioning a restriction endonuclease site between the fluorophore and quencher, a cleavage event causes permanent separation of the reporter from the quencher and causes a permanent increase in fluorescence in proportion to the amount of amplification product that accrues.

In this example a Tli I recognition sequence, “CTCGAG,” between the fluorophore and quencher in the primer/probe SEQ ID NO: 4 was subjected to Tli I restriction enzyme treatment. This enzyme is an extremely thermostable restriction endonuclease which could potentially provide for probe cleavage as amplification occurs in the same reaction mixture.

The following primer sets were evaluated in this example:

| SEQ. | | |
|---|---|---|
| ID | | |
| NO. | Sequence | Notes |
| 1 | CCAGCCGTAGTCGGTAGTAATCTATCAA | target |
| | GTTCTCATCGAAGCGGATAGGCGAGCG | |
| 2 | CCAGCCGTAGTCGGTAGT | PCR primer |
| | | “for” |
| 3 | CGCTCGCCTATCCGCTTC | PCR primer |
| | | “rev” |
| 4 | AQ-CCGTTCTCGAGTTtCGCTCGCCTAT | PCR probe- |
| | CCGCTTC | primer (rev) |

The oligonucleotides were the same as in Example 1.

PCR reaction mixtures had the following compositions in a 50 μl reaction volume:

### 10 mM TrisHCl pH 8.3,

### 50 mM KCl,

### 3 mM MgCl₂,

200 nM (each) dNTP
200 nM PCR primer “for”
200 nM PCR probe-primer/Rev
10⁸ copies Target DNA (SEQ ID NO: 1)
2.5 units AmpliTaq Gold DNA polymerase

PCR was done for 30 cycles but otherwise the temperature cycling conditions were the same as Example 1. In one reaction Tli I enzyme was added before PCR was carried out. In another reaction Tli I was also added after the PCR reaction. When Tli I was added after PCR, it was incubated in the PCR reaction mixture for 30 min at 75° C. The assays were also carried out as in Example 1 and show the result when no Tli I was added. To determine whether cleavage actually occurred, the products from each reaction were separated on a 10% polyacrylamide gel under denaturing conditions, stained using Gelstar™ stain, and visualized under an appropriate light.

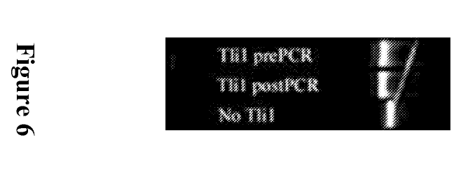

A photograph of the illuminated gel is provided in FIG. 6. FIG. 6 shows a gel having three lanes. From left to right the first two lanes show that cleavage occurred whether Tli I was added to reactions either before PCR (lane 1) or after PCR (lane 2). The third lane shows full length, uncleaved product when no Tli was added.

This example demonstrates that amplification can occur in the presence of Tli I restriction enzyme and shows that the enzyme can survive under PCR amplification conditions.

### EXAMPLE 5

This example demonstrates a method for determining suitable positions for a restriction enzyme recognition sequence between a fluorophore and quencher on the primer/probes of the invention. This example also specifically demonstrates suitable positions for the Tli I restriction enzyme recognition sequence that allows for cleavage of the probe by Tli I between the anthraquinone quencher at the 5′ terminus of the primer/probe and fluorescein dT.

The method involved creating a series of oligonucleotide primer/probes in which the position of the Tli I recognition sequence was varied with respect to the quencher and fluorophore. The oligonucleotides made for this example are listed below. The Tli I recognition sequence is shown in bold letters and the fluorescein-dT residue is designated with a t.

!SEQ.? ? ? ?!ID? ? Tli I?!NO.? Sequence? Cleavage

| | | |
|---|---|---|
| 16 | AQ-CCGTT [symbol] TCGCTCGCCTATCCGCTTC | No |
| 17 | AQ-T [symbol] CGCTCGCCTATCCGCTTC | No |
| 18 | AQ-TT [symbol] CGCTCGCCTATCCGCTTC | No |
| 19 | AQ-CCGTT [symbol] CGCTCGCCTATCCGCTTC | Yes |
| 20 | AQ-CCGTT [symbol] GTtCGCTCGCCTATCCGCTTC | Yes |
| 21 | AQ-CCGTT [symbol] GTTTtCGCTCGCCTATCCGC | Yes |
| | TTC | |
| 22 | AQ-CCGTT [symbol] GTTTTTtCGCTCGCCTATCCGC | Yes |
| | TTC | |
| 23 | AQ-CCGTTTT [symbol] GTTTTTtCGCTCGCCTATCC | Yes |
| | GCTTC | |

Oligonucleotides were prepared and purified as in Example 1. The oligonucleotides were annealed with complementary oligonucleotides to form duplex molecules and were then subjected to Tli I digestion according to the restriction enzyme manufacturer's instructions. The cleavage mixtures were separated on polyacrylamide gels by standard methods to determine cleavage efficiency.

As shown in the table, disruption of the cleavage sequence by a fluorescein labeled dT residue or positioning the recognition sequence within a seven to nine nucleotide spacing between the quencher and the fluorescein labeled dT blocks cleavage by Tli I. All sequences with a twelve base separation or greater (SEQ ID NOS: 15-20) were cleaved.

### EXAMPLE 6

This example evaluates whether a dual-labeled primer modified with a universal sequence on the 5′-end can be effectively coupled to a gene-specific amplification. The dual-labeled primer modified with the universal sequence was used in “real-time” PCR reactions and compared to “real-time” PCR reactions using a standard Taqman™ assay and the dual-labeled primer assay.

Reaction mixtures had the following composition:

### 10 mM TrisHCl pH 8.3,

### 50 mM KCl,

### 3 mM MgCl₂,

200 nM (each) dNTP
200 nM each primer
10⁶ cloned MP48 DNA
2.5 units AmpliTaq Gold DNA polymerase
(+/−3 μl, 30 units Tli I)
50 μl final reaction volume
The following primer sets were evaluated in this example using MP48-Amplicon (SEQ ID NO: 24):

| Primer Set | SEQ ID NO |
|---|---|
| Taqman assay | 25 |
| | 26 |
| Dual-labeled primer assay | 25 |
| | 27 |
| Universal primer assay | 25 |
| | 27 |
| | 28 |

The amplification was performed using the same procedure as Example 1, using 40 temperature cycles. Each system generated a fluorescent signal. The dual-labeled system and the Taqman® system had Ct values of 20, and the universal primer system's Ct value was 23. The lag in time for the universal primer is an expected inherent feature of the system due to the initial generation of targets from the unmodified bridge primer for use for the dual-labeled primer.

This example demonstrates that with the exception of the inherent lag time, the universal primer system is as effective as the Taqman® system or the dual-labeled primer system.

### EXAMPLE 7

This example evaluates the optimal concentration of the bridge primer by titrating the amount of the bridge primer and evaluating the fluorescence of each concentration. The procedures are the same as in Example 6 except there is no dual-labeled primer system, and there are multiple universal primer systems with the following concentrations:

100 nM bridge primer
10 nM
8 nM
4 nM
2 nM

The Taqman® system Ct value of 18½ Ct was still lower than the universal primer system values. The 100 nM, 10 nM and 8 nM concentrations all had similar Ct values around 21½. The 4 nM and the 2 nM concentrations had a Ct value around 23½.

This example demonstrates that the optimal concentration of the bridge primer in the universal primer system can range greatly from the standard 100 nM concentration and can be as low as 8 nM.

### EXAMPLE 8

This example demonstrates the effectiveness of the FQT assay illustrated in FIG. 1. The following sequences were prepared:

| SEQ | | |
|---|---|---|
| ID | | |
| NO | SEQUENCE | NOTES |
| 29 | GAACTCAGGCCAAGGTAGCGGAGGAGCTGGGCATG | Target |
| | CAGGAGTACGCCATAACCAACGACAAGACCAAGAG | |
| | GCCTGTGGCGCTTCGCACCAAGACCTTGGCGGACC | |
| | TTTTGGAATCATTTATTGCAGCGCTGTACATTGAT | |
| | AAGGATTTGGAATATGTTCATACTTTCATGAATGT | |
| | CTGCTTCTTTCCACGATTGAAAGAGTTCATTTTGA | |
| | ATCAGGATTGGAATGACCCCAAATCCCAGCTTCAG | |
| | CAGTGTTGCTTGACACTTAGGACAGAAGGAAAAGA | |
| | GCCAGACATTCCTCTGTACA | |
| 30 | ACCAACGACAAGACCAAGAG-HDrosha For1 | 5′- |
| 31 | TCGTGGAAAGAAGCAGACA-HDrosha Rev1 | nuclease |
| 32 | FAM-ACCAAGACCTTGGCGGACCTTT-IQ- | Assay |
| | HDrosha Probe 1 | |
| 33 | IQ-TTTTTTT [symbol] TTTTTTT (F-dT) | IQ-FAM |
| | ACCAACGACAAGACCAAGAG | |
| 34 | IQ-TTTTT [symbol] TTTTT (F-dT) | IQ-FAM |
| | ACCAACGACAAGACCAAGAG | |
| 35 | IQ-TTT [symbol] TTT (F-dT) | IQ-FAM |
| | ACCAACGACAAGACCAAGAG | |
| 36 | IQ-TTT [symbol] TTT (M-dT) | IQ-MAX |
| | ACCAACGACAAGACCAAGAG | |
| 37 | RQ-TTTTTTT [symbol] TTTTTTT (M-dT) | RQ-FAM |
| | ACCAACGACAAGACCAAGAG | |
| 38 | RQ-TTTTT [symbol] TTTTT (F-dT) | RQ-FAM |
| | ACCAACGACAAGACCAAGAG | |
| 39 | RQ-TTT [symbol] TTT (F-dT) | RQ-FAM |
| | ACCAACGACAAGACCAAGAG | |
| 40 | RQ-TTTCCTGGTTT (M-dT) | RQ-MAX |
| | ACCAACGACAAGACCAAGAG | |
| 41 | TCGGCTTCCTCCACGTCATC | Template |
| | | binding |
| | | domain |
| 42 | TCGTGGAAAGAAGCAGACA | Drosha |
| | | Rev |
| | | Primer |
| 43 | TCGGTTCCTCCACGTCATCTTCGTGGAAAGAAGCA | Chimeric |
| | GACA | Drosha |
| | | rev primer |
| 44 | IQ-TTTTTTT [symbol] TTTTTTT (F-dT) | Un- |
| | TTCGGCTTCCTCCACGTCAT (ddC) | modified |
| 45 | IQ-TTTTTTT [symbol] TTTTTTT (F-dT) | 5mC- |
| | TCGGCTTCCTCCACGTCAT (ddC) | |
| 46 | IQ-TTTTTTT [symbol] TTTTTTT (F-dT) | pdC- |
| | TCGGCTTCCTCCACGTCAT (ddC) | |
| 47 | IQ-TTTTTTT [symbol] TTTTTTT (F-dT) | LNA- |
| | TCGGCTTCCTCCACGTCAT (ddC) | |

IQ=Iowa Black azo quencher RQ=Iowa Black anthraquinone quencher
F=FAM fluorophore M=MAX fluorophore
Modified bases (underlined) include:
LNA=locked nucleic acid 5mc=5-methyl-dC pdC=propynyl-dC
Restriction sites are denoted in bold/italic

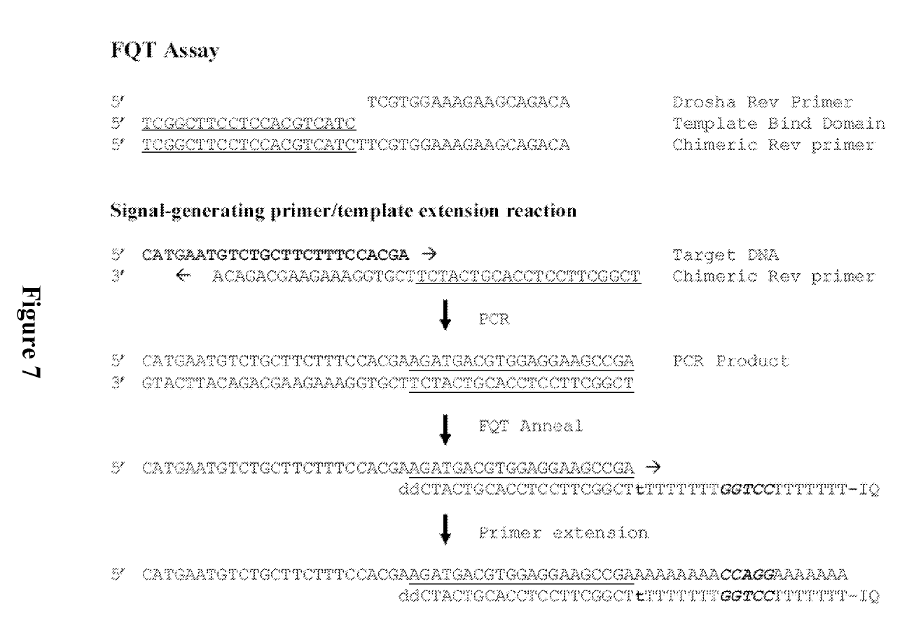

Multiple assays were carried out to compare the performance of FQ and FQT probes using the method of the invention. Sequence ID Nos. 30-32 were designed for a 5′ nuclease (Taqman®) assay. SEQ ID NOS: 33-40 were designed for use in the FQ assay format. SEQ ID NOS: 41-43 were designed for use in the FQT assay format. FIG. 7 illustrates sequence alignment of the biochemical events that take place during the FQT assay. SEQ ID NOS: 44-47 are unmodified or modified FQT template probes. The FQT reaction mixture contains the following:

### FQT Assay

0.25 U BioRad iTaq DNA polymerase
200 nM For primer SEQ ID NO: 30
200 nM Chimer Rev primer SEQ ID NO: 43

### 200 nM FQT-LNA SEQ ID NO: 47

### +/−10 U PspG1

### 10 mM MgCl₂

95^{3:00}−(95^{0:15}−63^{0:30}−72^{0:30})×45 cycles

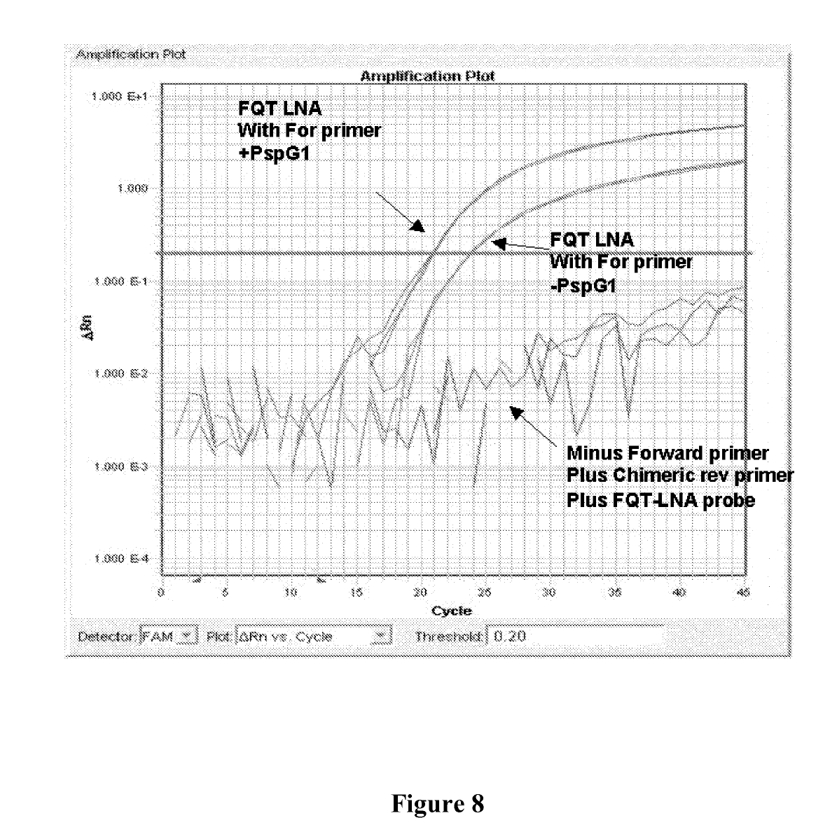

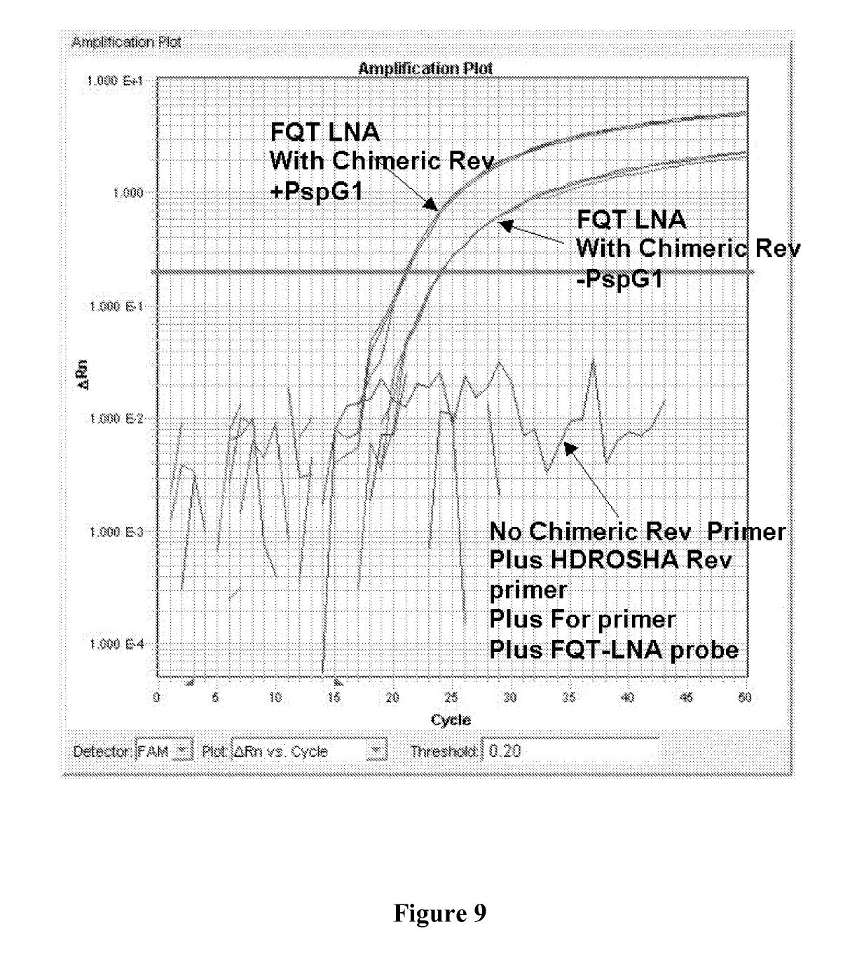

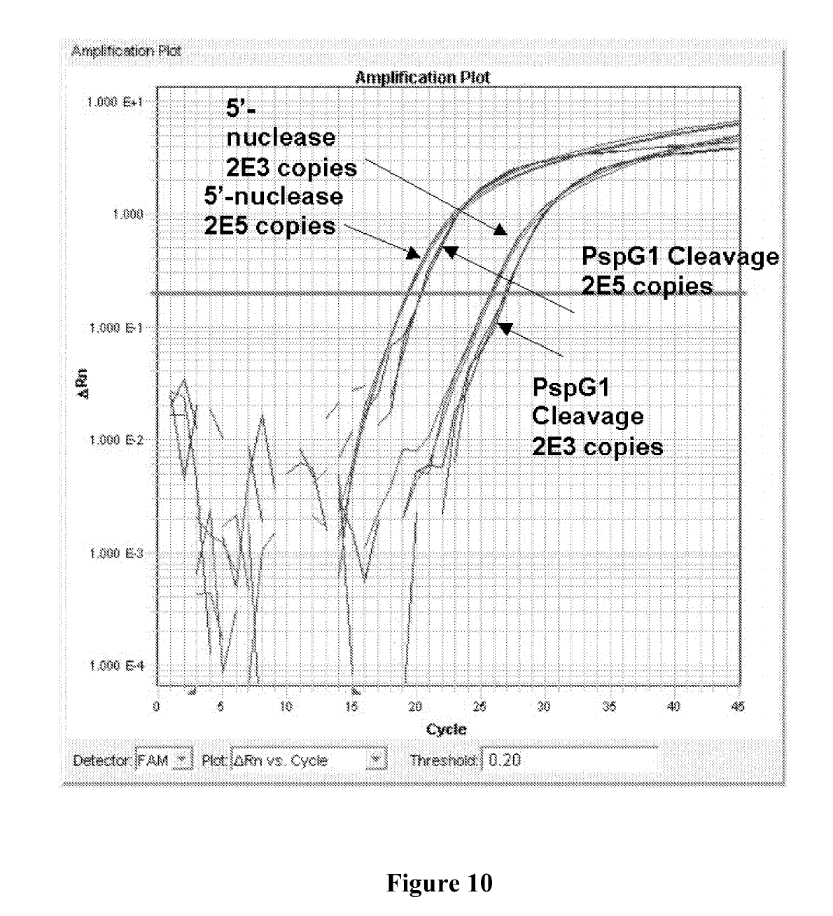

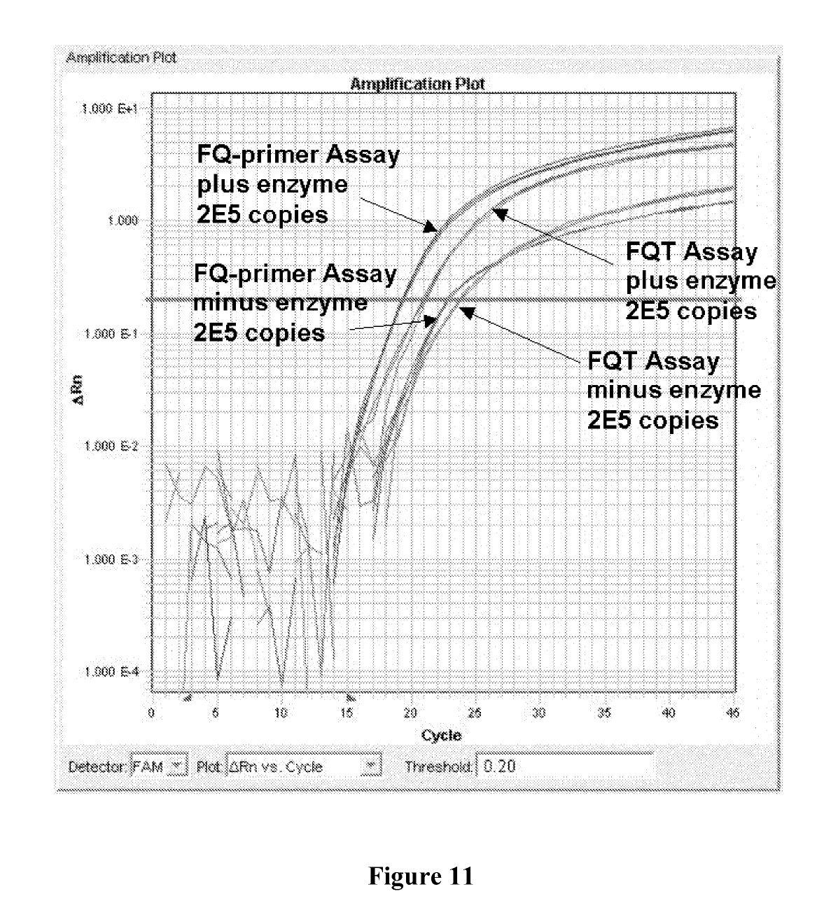

The FQT assay was performed on an AB7900 HT (Applied Biosystems) platform to determine if the assay would generate a signal with and without the presence of PspG1 enzyme. The amplification plots in FIGS. 8 and 9 show that the FQT probes with LNA modifications generate a fluorescence signal when all primer components are present; no signal is obtained when either primer is deleted. The FQT assay functioned in both cleavage and non-cleavage assay formats. Signal generation appeared ˜3 cycles earlier with probe cleavage. FIG. 10 compares the FQT assay (with and without cleavage) with the 5′ nuclease assay, and FIG. 11 compares the FQT assay with the FQ assay format (with and without cleavage). The FQT assay performed essentially identical with the 5′-nuclease assay and the FQ assay but showed a 1 cycle delay in signal generation, which is expected due to the assay design where the first signal generating event begins with the second cycle of PCR (FIG. 1 and FIG. 7).

The 5′-nuclease reaction mixture was as follows:

0.25 U BioRad iTaq DNA Polymerase
200 nM Rev primer SEQ ID NO: 31
200 nM For primer SEQ ID NO: 30
200 nM FAM-FQ probe SEQ ID NO: 32

### 3 mM MgCl₂

95^{3:00}−(95^{0:15}−63^{0:30}−72^{0:30})×45 cycles

The results of Example 8 demonstrate that the FQT assay has similar detection sensitivity as compared to either the FQ assay or the 5′-nuclease assay and should be functionally interchangeable for quantitative nucleic acid detection.

### EXAMPLE 9

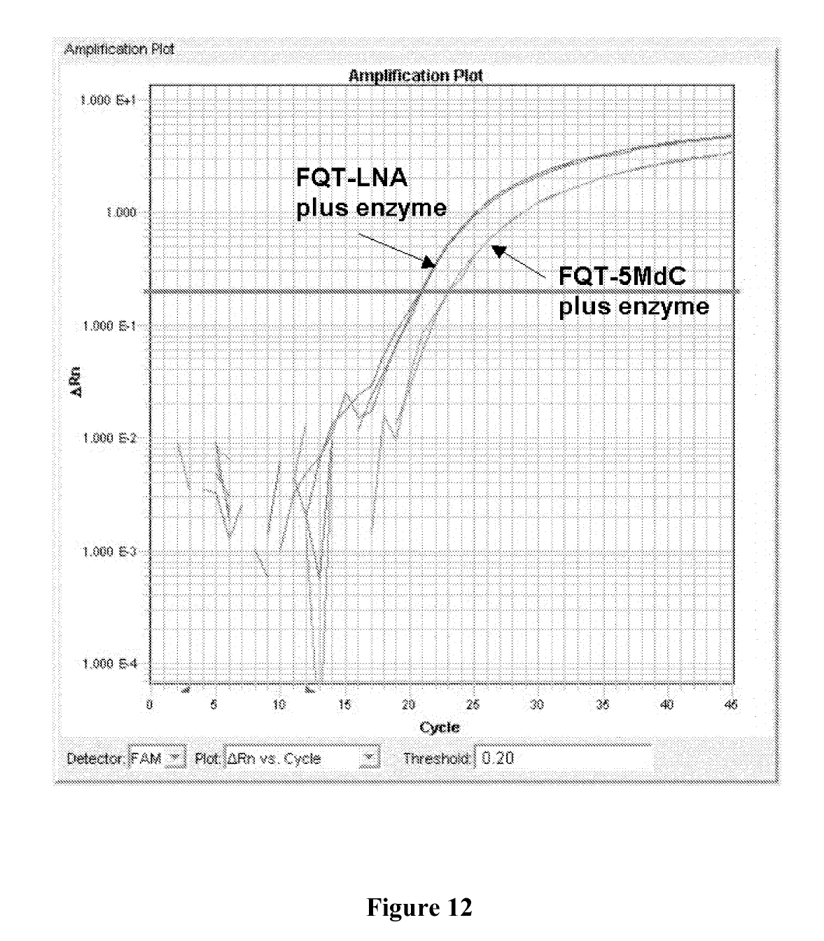

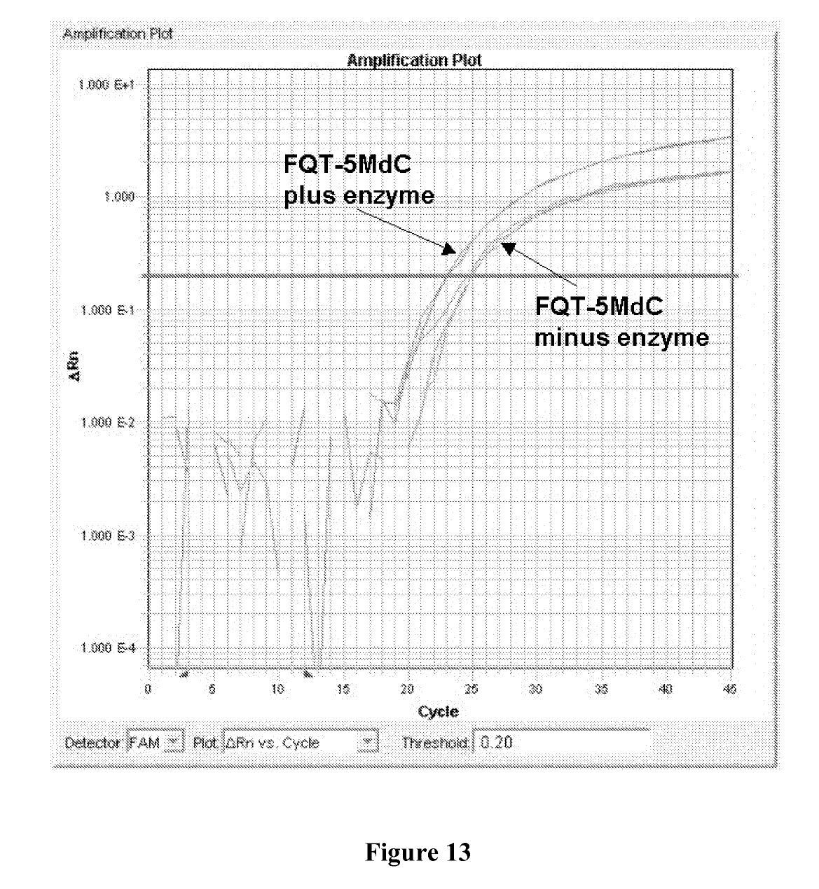

The following example offers a functional comparison of alternative FQT probe compositions. FQT probes (see SEQ ID NOS: 44-47) were either unmodified or modified with 5-methyl-dC, propynyl-dC or locked nucleic acid (LNA) bases. FIG. 12 shows the results of a comparison between an LNA-modified FQT probe with a 5-methyl-dC-modified FQT probe when a cleaving enzyme (PspG1) is present. FIG. 13 shows the same reactions function without probe cleavage.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A primer oligonucleotide for detecting a target nucleic acid sequence in a sample, the primer comprising:
a) a priming domain located on a 3′ end of the primer, wherein the priming domain has complementarity to the target nucleic acid sequence;
b) a reporter domain located on a 5′ end of the primer, wherein the reporter is non-complementary to the target and is modified to contain a fluorescence donor group and a fluorescence acceptor group; and
c) a cleaving element within the reporter domain positioned between the donor and the acceptor groups, wherein the cleaving element can specifically be cleaved when in double-strand form, wherein the double strand occurs via DNA synthesis using the reporter domain as a template.

2. The primer according to claim 1 wherein the cleaving element is a restriction endonuclease enzyme recognition site.

3. The primer according to claim 2 wherein the restriction endonuclease enzyme site is specific for a thermostable restriction endonuclease.

4. The primer according to claim 3 wherein the restriction endonuclease enzyme site is capable of being cleaved by PspG1.

5. The primer according to claim 3 wherein the restriction endonuclease enzyme site is capable of being cleaved by Tli I.

6. The primer according to claim 1 wherein the cleaving element is a ribonuclease enzyme recognition site.

7. The primer according to claim 6 wherein the ribonuclease enzyme recognition site is capable of being cleaved by an RNase H.

8. The primer according to claim 6 wherein the ribonuclease enzyme recognition site is capable of being cleaved by a thermostable RNase H.

9. The primer according to claim 8 wherein the thermostable RNase H is RNase H II from Pyrococcus kodakaraensis.

10. The primer according to claim 1 wherein the cleaving element is a single ribonucleotide recognized by a ribonuclease enzyme capable of cleaving a heteroduplex containing a single ribonucleotide.

11. The primer according to claim 1 wherein the sample is from an amplification assay.

12. The primer according to claim 1 wherein the sample is from a PCR assay.

13. The primer according to claim 1 wherein the sample is from a polynomial amplification assay.

14. A primer for detecting a target nucleic acid sequence in a sample, the oligonucleotide comprising:
a) a primer domain located on a 3′ end of the oligonucleotide, wherein the primer has complementarity to the target nucleic acid sequence;
b) a reporter domain located on a 5′ end of the nucleotide, wherein the reporter is non-complementary to the target and is modified to contain a fluorescence donor group and a fluorescence acceptor group; and
c) a configuration within the reporter domain, wherein the physical distance between the fluorophore and the quencher groups will increase when the primer shifts from single-stranded to double-stranded conformation, wherein the cleaving element can specifically be cleaved when in double-strand form, wherein the double strand occurs via DNA synthesis using the reporter domain as a template.

15. The primer according to claim 14 wherein the sample is from an amplification assay.

16. The primer according to claim 14 wherein the sample is from a PCR assay.

17. The primer according to claim 14 wherein the sample is from a polynomial amplification assay.

18. A template oligonucleotide for detecting a target nucleic acid sequence in a sample, the template oligonucleotide comprising:
a) a binding domain located on a 3′ end of the template oligonucleotide, wherein the binding domain comprises a sequence that is identical to a second binding domain on the 5′-end of a chimeric target-specific amplification primer, said 5′-end of a chimeric target-specific amplification primer domain being non-complementary to the target nucleic acid;
b) a reporter domain located on a 5′ end of the template oligonucleotide, wherein the reporter has a non-complementary sequence to the target sequence and the reporter is modified to contain a fluorophore group and a quencher group; and
c) a cleaving element within the reporter between the fluorophore and the quencher, wherein an enzyme that is able to cleave a double-stranded nucleic acid will cleave the template oligonucleotide at the cleaving element when the oligonucleotide binds with the target nucleic acid sequence; and
d) a 3′-terminal blocking group which prevents the template oligonucleotide from itself functioning as a primer.

19. The template oligonucleotide according to claim 18 wherein the cleaving element is a restriction endonuclease enzyme recognition site.

20. The template oligonucleotide according to claim 19 wherein the restriction endonuclease enzyme site is specific for a thermostable restriction endonuclease.

21. The template oligonucleotide according to claim 20 wherein the restriction endonuclease enzyme site is capable of being cleaved by PspG1.

22. The template oligonucleotide according to claim 20 wherein the restriction endonuclease enzyme site is capable of being cleaved by Tli I.

23. The template oligonucleotide according to claim 18 wherein the cleaving element is a ribonuclease enzyme recognition site.

24. The template oligonucleotide according to claim 23 wherein the ribonuclease enzyme recognition site is capable of being cleaved by an RNase H.

25. The template oligonucleotide according to claim 24 wherein the ribonuclease enzyme recognition site is capable of being cleaved by a thermostable RNase H.

26. The template oligonucleotide according to claim 25 wherein the thermostable RNase H is RNase H II from Pyrococcus kodakaraensis.

27. The template oligonucleotide according to claim 18 wherein the cleaving element is a single ribonucleotide recognized by a ribonuclease enzyme capable of cleaving a heteroduplex containing a single ribonucleotide.

28. The template oligonucleotide according to claim 18 wherein the sample is from an amplification assay.

29. The template oligonucleotide according to claim 18 wherein the sample is from a PCR assay.

30. The template oligonucleotide according to claim 18 wherein the sample is from a polynomial amplification assay.

31. A template oligonucleotide for detecting a target nucleic acid sequence in a sample, the oligonucleotide comprising:
a) a binding domain located on a 3′ end of the oligonucleotide, wherein the binding domain comprises a sequence that is identical to a binding domain on the 5′-end of a chimeric target-specific amplification primer, said 5′-end of a chimeric target-specific amplification primer domain being non-complementary to the target nucleic acid;
b) a reporter domain located on a 5′ end of the template nucleotide, wherein the reporter has a non-complementary sequence to the target sequence and the reporter is modified to contain a fluorophore group and a quencher group; and
c) a configuration within the reporter domain, wherein the physical distance between the fluorophore and the quencher groups will increase when the primer shifts from single-stranded to double-stranded conformation, wherein the double strand occurs via DNA synthesis using the reporter domain as a template;
d) a 3′-terminal blocking group which prevents the oligonucleotide from itself functioning as a primer.

32. The template oligonucleotide according to claim 31 wherein the sample is from an amplification assay.

33. The template oligonucleotide according to claim 31 wherein the sample is from a PCR assay.

34. The template oligonucleotide according to claim 31 wherein the sample is from a polynomial amplification assay.

35. A method for detecting a target nucleic acid sequence in a sample, the method comprising:
a) providing a first oligonucleotide containing a primer domain on a 3′ end of the oligonucleotide and a reporter domain on a 5′ end of the oligonucleotide, wherein the primer is complementary to the nucleic acid sequence;
b) providing a second oligonucleotide in reverse orientation to the first oligonucleotide that together can function to prime an amplification reaction on said target nucleic acid;
c) heating a mixture containing the nucleic acid to denature double-stranded structures and cooling the mixture to permit annealing of the primers to the target nucleic acid;
d) synthesizing new nucleic acid strands using DNA polymerase, wherein the new nucleic acids will be complementary to template single strand structures, including the primer and the reporter domains of the first primer;
e) repeating steps (c)-(d) wherein a plurality of the new strand nucleic acid will be synthesized, and the new strand nucleic acid will form a duplex with a second new strand nucleic acid; and
f) detecting a change in fluorescence signal caused by the conformation change from a single-stranded to a double-stranded structure.

36. The method of claim 35 wherein the change in fluorescence signal caused by the conformation change from the single-stranded to the double-stranded structure is caused is due to a spatial separation between a fluorophore and a quencher located on the reporter domain.

37. The method of claim 35 wherein the change in fluorescence signal caused by the conformation change from the single-stranded to the double-stranded structure is caused is due to a cleavage within the reporter domain.

38. The method according to claim 37 wherein the cleavage is through the use of a restriction endonuclease enzyme recognition site.

39. The method according to claim 38 wherein the restriction endonuclease enzyme site is specific for a thermostable restriction endonuclease.

40. The method according to claim 38 wherein the restriction endonuclease enzyme site is capable of being cleaved by PspG1.

41. The method according to claim 38 wherein the restriction endonuclease enzyme site is capable of being cleaved by Tli I.

42. The method according to claim 37 wherein the cleaving element is a ribonuclease enzyme recognition site.

43. The method according to claim 42 wherein the ribonuclease enzyme recognition site is capable of being cleaved by an RNase H.

44. The method according to claim 42 wherein the ribonuclease enzyme recognition site is capable of being cleaved by a thermostable RNase H.

45. The method according to claim 44 wherein the thermostable RNase H is RNase H II from Pyrococcus kodakaraensis.

46. The method according to claim 37 wherein the cleavage occurs at a single ribonucleotide recognized by a ribonuclease enzyme capable of cleaving a heteroduplex containing a single ribonucleotide.

47. A method for detecting a target nucleic acid sequence in a sample, the method comprising:
a) providing the primer oligonucleotide from claim 1;
b) providing a second oligonucleotide in reverse orientation to the first oligonucleotide that together can function to prime an amplification reaction on said target nucleic acid;
c) heating a mixture containing the nucleic acid to denature double-stranded structures and cooling the mixture to permit annealing of the primers to the target nucleic acid;
d) synthesizing new nucleic acid strands using DNA polymerase, wherein the new nucleic acids will be complementary to template single strand structures, including the primer and the reporter domains of the first primer;
e) repeating steps (c)-(d) wherein a plurality of the new strand nucleic acid will be synthesized, and the new strand nucleic acid will form a duplex with a second new strand nucleic acid; and
f) detecting a change in fluorescence signal caused by the conformation change from a single-stranded to a double-stranded structure

48. A method for detecting a target nucleic acid sequence in a sample, the method comprising:
a) providing a first oligonucleotide containing a primer domain on a 3′ end of the first oligonucleotide and a template binding domain on a 5′ end of the first oligonucleotide, wherein the primer is complementary to the target nucleic acid sequence and the template binding domain on the 5′ end of the first oligonucleotide is non-complementary to the target nucleic acid sequence;
b) separating the target nucleic acid sequence into a target single strand structure;
c) annealing the primer to the target single strand structure;
d) synthesizing a second strand nucleic acid, wherein the second strand nucleic acid will be complementary to the target single strand structure and the primer;
e) separating the second strand nucleic acid;
f) annealing a template oligonucleotide containing a primer-specific binding domain on the 3′ end and a reporter domain on the 5′ end of the second oligonucleotide, wherein the primer binding domain is complementary to the second strand nucleic acid synthesized above but is non-complementary to the original target nucleic acid;
g) synthesizing a third strand nucleic acid using said second strand nucleic acid as primer, wherein the third strand nucleic acid will include the second strand nucleic acid structure, and a domain complementary to the reporter of the template of the primer-binding domain, wherein DNA synthesis causes the template nucleic acid to form duplex structure, causing a conformational change which enables a detection event to occur;
h) separating the third strand nucleic acid;
f) repeating steps (g)-(h) wherein a plurality of the third strand nucleic acid will be synthesized, and the third strand nucleic acid will form a duplex with a fourth strand nucleic acid.

49. The method of claim 48 wherein the change in fluorescence signal caused by the conformation change from the single-stranded to the double-stranded structure is caused is due to a spatial separation between a fluorophore and a quencher located on the reporter domain.

50. The method of claim 48 wherein the change in fluorescence signal caused by the conformation change from the single-stranded to the double-stranded structure is caused is due to a cleavage within the reporter domain.

51. The method according to claim 50 wherein the cleavage is through the use of a restriction endonuclease enzyme recognition site.

52. The method according to claim 51 wherein the restriction endonuclease enzyme site is specific for a thermostable restriction endonuclease.

53. The method according to claim 51 wherein the restriction endonuclease enzyme site is capable of being cleaved by PspG1.

54. The method according to claim 51 wherein the restriction endonuclease enzyme site is capable of being cleaved by Tli I.

55. The method according to claim 50 wherein the cleaving element is a ribonuclease enzyme recognition site.

56. The method according to claim 55 wherein the ribonuclease enzyme recognition site is capable of being cleaved by an RNase H.

57. The method according to claim 55 wherein the ribonuclease enzyme recognition site is capable of being cleaved by a thermostable RNase H.

58. The method according to claim 57 wherein the thermostable RNase H is RNase H II from Pyrococcus kodakaraensis.

59. The method according to claim 50 wherein the cleavage occurs at a single ribonucleotide recognized by a ribonuclease enzyme capable of cleaving a heteroduplex containing a single ribonucleotide.

60. A method for detecting a target nucleic acid sequence in a sample, the method comprising:
a) providing a first oligonucleotide containing a primer domain on a 3′ end of the first oligonucleotide and a template binding domain on a 5′ end of the first oligonucleotide, wherein the primer is complementary to the target nucleic acid sequence and the template binding domain on the 5′ end of the first oligonucleotide is non-complementary to the target nucleic acid sequence;
b) separating the target nucleic acid sequence into a target single strand structure;
c) annealing the primer to the target single strand structure;
d) synthesizing a second strand nucleic acid, wherein the second strand nucleic acid will be complementary to the target single strand structure and the primer;
e) separating the second strand nucleic acid;
f) annealing a template oligonucleotide, said template oligonucleotide comprising;
i. a binding domain located on a 3′ end of the oligonucleotide, wherein the binding domain comprises a sequence that is identical to a binding domain on the 5′-end of a chimeric target-specific amplification primer, said 5′-end of a chimeric target-specific amplification primer domain being non-complementary to the target nucleic acid;
ii. a reporter domain located on a 5′ end of the template nucleotide, wherein the reporter has a non-complementary sequence to the target sequence and the reporter is modified to contain a fluorophore group and a quencher group; and
iii. a separation element within the reporter between the donor and the acceptor, wherein separation occurs; and
iv. a 3′-terminal blocking group which prevents the oligonucleotide from itself functioning as a primer;
g) synthesizing a third strand nucleic acid using said second strand nucleic acid as primer, wherein the third strand nucleic acid will include the second strand nucleic acid structure, and complementary to the reporter of the template of the primer-binding domain, and the reporter domain, wherein DNA synthesis causes the template nucleic acid to form duplex structure, causing a conformational change which enables a detection event to occur;
h) separating the third strand nucleic acid;
f) repeating steps (g)-(h) wherein a plurality of the third strand nucleic acid will be synthesized, and the third strand nucleic acid will form a duplex with a fourth strand nucleic acid.

61. The method according to claim 60 wherein the detection event occurs because of a physical separation of a fluorophore and a quencher on the reporter domain via a cleavage event.

62. The method according to claim 60 wherein the separation is due to the increase in distance between fluorophore and the quencher as a result of duplex formation.

63. The method according to claim 60 wherein the separation is due to a cleaving of the reporter domain between the fluorophore and the quencher.

64. The method according to claim 63 wherein the cleaving of the reporter domain is caused by a restriction endonuclease enzyme.

65. The method according to claim 64 wherein the cleaving of the reporter domain is caused by a thermostable restriction endonuclease enzyme.

66. The method according to claim 63 wherein the cleaving of the reporter domain is caused by a ribonuclease enzyme.

67. The method according to claim 63 wherein the cleaving of the reporter domain is caused by RNase H.

68. The method according to claim 63 wherein the cleaving of the reporter domain is caused by a thermostable RNase H.

69. The method according to claim 68 wherein the thermostable RNase H is RNase H II from Pyrococcus kodakaraensis.

70. A method for detecting presence of a target sequence comprising:
a) hybridizing to the target sequence a signal primer comprising a target binding sequence and a ribonuclease recognition sequence 5′ to the target binding sequence, the ribonuclease recognition sequence flanked by a donor fluorophore and an acceptor dye such that fluorescence of the donor fluorophore is quenched;
b) in a primer extension reaction, synthesizing a complementary strand using the signal primer as a template, thereby rendering the ribonuclease recognition sequence double-stranded;
c) cleaving or nicking the double-stranded ribonuclease recognition sequence with a ribonuclease, thereby reducing donor fluorophore quenching and producing a change in a fluorescence parameter, and;
d) detecting the change in the fluorescence parameter as an indication of the presence of the target sequence.


SEQUENCE LISTING (47 sequences)

SEQ ID NO: 1 - DNA, 55 bp, Artificial
  Synthetic amplicon to test system
ccagccgtagtcggtagtaatctatcaagttctcatcgaagcggataggcgagcg

SEQ ID NO: 2 - DNA, 18 bp, Artificial
  synthetic PCR forward primer
cgctcgcctatccgcttc

SEQ ID NO: 3 - DNA, 18 bp, Artificial
  Synthetic PCR Reverse Primer
ccagccgtagtcggtagt

SEQ ID NO: 4 - DNA, 31 bp, Artificial
  synthetic oligonucleotide probe
ccgttctcgagttcgctcgcctatccgcttc

SEQ ID NO: 5 - DNA, 24 bp, Artificial
  synthetic oligonucleotide probe
ccgtttcgctcgcctatccgcttc

SEQ ID NO: 6 - DNA, 25 bp, artificial
  synthetic oligonucleotide probe
ccgttctcgctcgcctatccgcttc

SEQ ID NO: 7 - DNA, 27 bp, Artificial
  synthetic oligonucleotide probe
ccgttctcgcgctcgcctatccgcttc

SEQ ID NO: 8 - DNA, 29 bp, Artificial
  synthetic oligonucleotide probe
ccgttctcgagcgctcgcctatccgcttc

SEQ ID NO: 9 - DNA, 31 bp, Artificial
  synthetic oligonucleotide probe
ccgttctcgaggtcgctcgcctatccgcttc

SEQ ID NO: 10 - DNA, 33 bp, Artificial
  synthetic oligonucleotide probe
ccgttctcgaggtttcgctcgcctatccgcttc

SEQ ID NO: 11 - DNA, 35 bp, Artificial
  synthetic oligonucleotide probe
ccgttctcgaggtttttcgctcgcctatccgcttc

SEQ ID NO: 12 - DNA, 37 bp, Artificial
  synthetic oligonucleotide probe
ccgttttctcgaggtttttcgctcgcctatccgcttc

SEQ ID NO: 13 - DNA, 27 bp, Artificial
  synthetic oligonucleotide probe
ccgttctcgcgctcgcctatccgcttc

SEQ ID NO: 14 - DNA, 31 bp, Artificial; identical to SEQ ID NO: 9
  synthetic oligonucleotide probe

SEQ ID NO: 15 - DNA, 35 bp, Artificial; identical to SEQ ID NO: 11
  synthetic oligonucleotide probe

SEQ ID NO: 16 - DNA, 29 bp, Artificial
  synthetic oligonucleotide probe
ccgttccgagtcgctcgcctatccgcttc

SEQ ID NO: 17 - DNA, 25 bp, Artificial
  synthetic oligonucleotide probe
tctcgagcgctcgcctatccgcttc

SEQ ID NO: 18 - DNA, 26 bp, Artificial
  synthetic oligonucleotide probe
ttctcgagcgctcgcctatccgcttc

SEQ ID NO: 19 - DNA, 29 bp, Artificial
  synthetic oligonucleotide probe
ccgttctcgagcgctcgcctatccgcttc

SEQ ID NO: 20 - DNA, 31 bp, Artificial; identical to SEQ ID NO: 9
  synthetic oligonucleotide probe

SEQ ID NO: 21 - DNA, 33 bp, Artificial; identical to SEQ ID NO: 10
  synthetic oligonucleotide probe

SEQ ID NO: 22 - DNA, 35 bp, Artificial; identical to SEQ ID NO: 11
  synthetic oligonucleotide probe

SEQ ID NO: 23 - DNA, 37 bp, Artificial
  synthetic oligonucleotide probe
ccgttctcgaggtttttttcgctcgcctatccgcttc

SEQ ID NO: 24 - DNA, 76 bp, Artificial
  MP48
cagaaggttatcatctgccatcgaggcacccgttcaccctcccccagtgacccggattatggtctccctcctcttg

SEQ ID NO: 25 - DNA, 24 bp, Artificial
  MP48
cagaaggttatcatctgccatcga

SEQ ID NO: 26 - DNA, 23 bp, Artificial
  MP48
caagaggagggagaccataatcc

SEQ ID NO: 27 - DNA, 37 bp, Artificial
  MP48
ccgttctcgaggtcagaaggttatcatctgccatcga

SEQ ID NO: 28 - DNA, 41 bp, Artificial
  MP48
cgctcgcctatccgcttcagaaggttatcatctgccatcga

SEQ ID NO: 29 - DNA, 300 bp, Artificial
  Target
gaactcaggccaaggtagcggaggagctgggcatgcaggagtacgccataaccaacgacaagaccaagaggcctgtggcgcttcgcaccaagaccttggcggaccttttggaatcatttattgcagcgctgtacattgataaggatttggaatatgttcatactttcatgaatgtctgcttctttccacgattgaaagagttcattttgaatcaggattggaatgaccccaaatcccagcttcagcagtgttgcttgacacttaggacagaaggaaaagagccagacattcctctgtaca

SEQ ID NO: 30 - DNA, 20 bp, Artificial
  HDrosha For1
accaacgacaagaccaagag

SEQ ID NO: 31 - DNA, 38 bp, Artificial
  HDrosha Rev1
tcgtggaaagaagcagacatgtctgcttctttccacga

SEQ ID NO: 32 - DNA, 22 bp, Artificial
  HDrosha Probe 1
accaagaccttggcggaccttt

SEQ ID NO: 33 - DNA, 40 bp, Artificial
  Synthetic oligonucleotide probe
tttttttcctggttttttttaccaacgacaagaccaagag

SEQ ID NO: 34 - DNA, 36 bp, Artificial
  synthetic oligonucleotide probe
tttttcctggttttttaccaacgacaagaccaagag

SEQ ID NO: 35 - DNA, 32 bp, Artificial
  synthetic oligonucleotide probe
tttcctggttttaccaacgacaagaccaagag

SEQ ID NO: 36 - DNA, 32 bp, Artificial; identical to SEQ ID NO: 35
  synthetic oligonucleotide probe

SEQ ID NO: 37 - DNA, 40 bp, Artificial; identical to SEQ ID NO: 33
  synthetic oligonucleotide probe

SEQ ID NO: 38 - DNA, 36 bp, Artificial; identical to SEQ ID NO: 34
  synthetic oligonucleotide probe

SEQ ID NO: 39 - DNA, 32 bp, Artificial; identical to SEQ ID NO: 35
  synthetic oligonucleotide probe

SEQ ID NO: 40 - DNA, 32 bp, Artificial; identical to SEQ ID NO: 35
  synthetic oligonucleotide probe

SEQ ID NO: 41 - DNA, 20 bp, Artificial
  Template binding domain
tcggcttcctccacgtcatc

SEQ ID NO: 42 - DNA, 19 bp, Artificial
  Drosha Rev Primer
tcgtggaaagaagcagaca

SEQ ID NO: 43 - DNA, 40 bp, Artificial
  chimeric drosha rev primer
tcggcttcctccacgtcatcttcgtggaaagaagcagaca

SEQ ID NO: 44 - DNA, 40 bp, Artificial
  Unmodified FQT probe
tttttttcctggtttttttttcggcttcctccacgtcatc

SEQ ID NO: 45 - DNA, 41 bp, Artificial
  5mC-modified
tttttttcctggtttttttdttnggnttnntnnangtnatc

SEQ ID NO: 46 - DNA, 41 bp, Artificial; identical to SEQ ID NO: 45
  pdC-modified FQT probe

SEQ ID NO: 47 - DNA, 41 bp, Artificial; identical to SEQ ID NO: 45
  LNA modified